(19) United States
(12) Patent Application Publication
(10) Pub. No.: US 2024/0245811 A1
(43) Pub. Date: Jul. 25, 2024

(54) ALPHA RADIOLABELED GASTRIN ANALOGUE AND USE THEREOF IN METHODS OF TREATING CCKB RECEPTOR POSITIVE DISEASES

(71) Applicant: Paul Scherrer Institut, Villigen PSI (CH)

(72) Inventors: Michal Grzmil, Basel (CH); Roger Schibli, Baden (CH); Martin Behe, Gelterkinden (CH); Alain Blanc, Aarau (CH); Yun Qin, Ennetbaden (CH)

(21) Appl. No.: 18/040,021

(22) PCT Filed: Jul. 30, 2021
(86) PCT No.: PCT/EP2021/071463

§ 371 Date: Jan. 31, 2023

(30) Foreign Application Priority Data
Jul. 31, 2020 (WO) PCT/EP2020/071730
Sep. 17, 2020 (WO) PCT/EP2020/075956

(51) Int. Cl. A61K 51/08; A61P 35/00; C07K 7/08

(52) U.S. Cl. CPC A61K 51/088; A61P 35/00; C07K 7/08

(57) ABSTRACT

An alpha radiolabeled gastrin analogue is used in peptide receptor radionuclide therapy (PRRT) applications. In particular, an alpha radiolabeled gastrin analogue, which exhibits excellent biodistribution and therapeutic efficacy while toxicity for healthy tissues is prevented and/or reduced. The alpha radiolabeled gastrin analogue is used in methods of treating cholecystokinin (CCKB) receptor positive diseases.

### REFERENCE TO THE SEQUENCE LISTING

This application includes an electronically submitted sequence listing in .txt format. The .txt file contains a sequence listing entitled “2022P20050_ST25.txt” created on Jul. 14, 2023 and is 3,435 bytes in size. The sequence listing contained in this .txt file is part of the specification and is hereby incorporated by reference herein in its entirety.

### SPECIFICATION

The present invention relates to an alpha radiolabeled gastrin analogue and its use in peptide receptor radionuclide therapy (PRRT) applications. In particular, the present invention relates to an alpha radiolabeled gastrin analogue, e.g. a gastrin analogue labeled with actinium-255, which exhibits excellent biodistribution and therapeutic efficacy while toxicity for healthy tissues can be prevented and/or reduced. The present invention also relates to the use of an alpha radiolabeled gastrin analogue in methods of treating CCKB receptor positive diseases, e.g. tumors or cancers.

### FIELD AND BACKGROUND OF THE INVENTION

G-protein coupled receptors (GPCRs) constitute a superfamily of membrane proteins whose function is to transduce a chemical signal across the cell membrane. When a ligand binds to a GPCR, it causes a conformational change allowing the GPCR to activate and release associated G proteins, which subsequently triggers signal transduction pathways.

Overexpression of G-protein coupled receptors (GPCRs) that selectively bind their peptide ligands allow the development of peptide receptor radionuclide therapy (PRRT) for human cancers (Lappano et al. Nat Rev Drug Discov. 2011, 10(1), 47-60). One of the most important goal of PRRT is to achieve high uptake of radiolabeled ligands into target cells, e.g. cancer cells, thus leading to radiation-induced DNA damage and cell death. Therefore, strategies to increase the uptake of radiopharmaceuticals in target cells while sparing the surrounding healthy tissues from side effects have been considered.

GPCRs targeted by agonistic ligand-based therapeutics undergo conformational changes, which lead to the exchange of GDP for GTP on the G-protein alpha subunit (Gα). Subsequent dissociation of the Gα and Gβγ subunits from the receptor results in activation of various kinase signaling pathways involving protein kinases A and C (PKA; PKC) as well as phosphoinositide 3-kinase (PI3K) and mitogen activated protein kinases (MAPKs) (O'Hayre et al. Curr Opin Cell Biol. 2014, 27, 126-135). Subsequently, activated GPCRs undergo desensitization via an arrestin-mediated internalization process, whereby GPCRs can be trafficked to lysosomes for degradation, or to endosomes for their recycling back to the cell surface (Rajagopal et al. Cell Signal. 2018, 41, 9-16). This internalization process enables the delivery of ligand-conjugated radioactive nuclides into target cells, e.g. cancer cells.

Medullary thyroid cancer (MTC) is a neuroendocrine tumor derived from calcitonin-producing C cells. Accounting for 3-5% of all thyroid cancers, MTC is a relatively rare cancer entity (Hadoux et al. Lancet Diabetes Endocrinol. 2016, 4(1), 64-71). Unfortunately, responses to conventional chemotherapy (usually doxorubicin alone or in combination with cisplatin) are only transient and benefit is limited to a small number of patients. In addition, MTC cells do not accumulate iodine and thus, do not respond to radioactive iodine treatment (Verburg et al. Methods. 201, 55(3), 230-237). Currently, MTC accounts for 14% of all thyroid cancer-related deaths, indicating the need for better treatments especially in metastasized patients (Roman et al. Cancer. 2006, 107(9), 2134-2142).

Small-cell lung cancer (SCLC) is a highly malignant cancer that most commonly arises in the lung. It usually presents large, rapidly developing lesions arising from the centrally located tracheobronchial airways and invading the mediastinum. For one third of patients diagnosed with SCLC at a limited-stage of disease, chemoradiotherapy leads to a cure rate of approximately 25%. On the other hand, both extensive-stage and relapsed SCLC are often considered incurable and available treatments, e.g. chemotherapy, are usually administered with a palliative intent. The prognosis of patients with relapsed SCLC remains dismal, with a median overall survival of about 6 months (Travis et al. J Thorac Oncol. 2015, 10(9), 1243-1260).

Extrapulmonary small-cell carcinoma (EPSCC) refers to small-cell carcinomas arising outside the lungs. They most commonly develop in the gastrointestinal and genitourinary systems. EPSCCs are rare neoplasms constituting only 2.5% to 5.0% of all small-cell carcinoma cases and 0.1% to 0.4% of all cancers. EPSCC has an aggressive natural history characterized by rapid local progression, early widespread metastases, and recurrence following treatment. The prognosis of patients diagnosed with EPSCC is relatively poor despite chemotherapy, with median survival ranging from 3 to 27 months and overall 5-year survival rates around 13% (Nakazawa et al. Oncol Lett. 2012, 4(4), 617-620).

High expression of cholecystokinin B receptor (CCKBR, sometimes also referred to as CCK2R), which belongs to the GPCR family, has been validated in a variety of cancers including MTC, gliomas, SCLC, colon cancer, ovarian cancer etc. (Reubi et al. Cancer Res. 1997, 57(7), 1377-1386). Furthermore, the small peptide hormone “minigastrin” is known to bind CCKBR with high affinity. Therefore, previous studies have suggested the use of radiolabeled gastrin analogues for PRRT, in particular for “theranostics” (therapy and diagnostics) applications.

Behr et al. have proposed the use of minigastrin analogues comprising a chelating moiety (DTPA) with good stability for various radionuclides, including beta- (e.g. ⁹⁰Y, ¹⁵³Sm), Auger electron- (e.g. ¹¹¹In, ⁶⁷Ga) and alpha-emitters (e.g. ²¹³Bi, ²²⁵Ac). However, it has been found that the radiolabeled gastrin analogues tend to accumulate in healthy tissues endogenously expressing CCKBR, especially in the stomach, irrespective of the radionuclide employed. This accumulation can lead to adverse side effects (toxicity) in healthy tissues such as hemorrhagic gastritis and, consequently, it may limit the dose of radiation which can be administered to a subject in need of treatment (J Nucl Med. 2001, 42(5):68P; Semin Nucl Med. 2002, 32(2), 97-109).

WO 2015/067473 A1 describes a gastrin analogue of formula DOTA-(DGlu)₆-Ala-Tyr-Gly-Trp-Nle-Asp-Phe-NH₂ (SEQ ID NO: 4)(PP-F11N) labeled with ¹⁷⁷Lu (in the following “¹⁷⁷Lu-PP-F11N”), which exhibits good tumor uptake as well as low accumulation in the kidneys, i.e. a good “tumor-to-kidney ratio”. Yet, in recent studies, it has been found that this compound can accumulate in healthy tissues (i.e. in the stomach) due to their endogenous CCKBR expression, thereby limiting the radiation dose and/or leading to adverse side effects (Sauter et al. J Nucl Med. 2019, 60(3), 393-399; Rottenburger et al. J Nucl Med. 2020, 61(4), 520-526). Furthermore, it has been found that lutetium-177 may cause damage to surrounding healthy cells due to its long tissue penetration range (cross-fire effect) and exhibit limited efficacy due to insufficient DNA damage especially in treating small-size neoplasms and/or diseases at an advanced (metastatic) stage such as (micro)metastases.

Having regard to the above, it is an object of the present invention to provide a compound for PRRT applications having excellent biodistribution and therapeutic efficacy as well as low toxicity for healthy tissues. It is a further object to provide a compound that can be used in methods of treating CCKB receptor positive diseases, in particular CCKB receptor positive diseases at an advanced stage.

### SUMMARY OF THE INVENTION

The present invention provides a compound, i.e. an alpha radiolabeled gastrin analogue, having excellent biodistribution and therapeutic efficacy as well as low toxicity (or no toxicity) for healthy tissues. In particular, the present inventors have found that the labeling of a specific gastrin analogue with an alpha radionuclide allows to achieve excellent biodistribution and therapeutic efficacy while adverse side effects due to accumulation of the radioactive compound in healthy tissues endogenously expressing CCKBR (e.g. in the stomach) and to irradiation of healthy tissues surrounding the target cells can be prevented and/or reduced. These findings are particularly surprising because alpha radionuclide-labeled gastrin compounds have been associated with increased toxicity in healthy tissues, especially in the stomach due its high endogenous CCKBR expression.

The present invention thus relates to an alpha radiolabeled gastrin analogue having the following formula (1):

(1)
(SEQ ID NO: 5)
Y-(DGlu)₆-Ala-Tyr-Gly-Trp-Axx-Asp-Phe-NH₂

wherein,
Axx represents an amino acid isosteric with methionine, and
Y represents a moiety that chelates an alpha radionuclide.

The compound of the present invention can be used in PRRT applications. The present invention thus also relates to an alpha radiolabeled gastrin analog for use in methods of treating CCKBR positive diseases, in particular GC, PADC, SCLC, EPSCC, MTC, gliomas, GEP-NETs, colon cancer, ovarian cancer, breast cancer, and any CCKB receptor positive diseases.

The present invention in particular includes the following embodiments (“Items”):
1. An alpha radiolabeled gastrin analogue having the following formula (1):

(1)
(SEQ ID NO: 5)
Y-(DGlu)₆-Ala-Tyr-Gly-Trp-Axx-Asp-Phe-NH₂

wherein,
Axx represents an amino acid isosteric with methionine, and
Y represents a moiety that chelates an alpha radionuclide.
2. The alpha radiolabeled gastrin analogue of item 1, wherein Axx represents an amino acid selected from the group consisting of isoleucine (Ile), norleucine (Nle), 2-amino-5-heptenoic acid, homo-norleucine (homo-Nle), homo-cysteine (homo-Cys), 2-amino-4-methoxybutanoic acid, telluromethionine (Te-Met), selenomethionine (Se-Met) and phenylglycine (Phg), preferably Ne.
3. The alpha radiolabeled gastrin analogue of item 1 or 2, wherein Y is a moiety derived from 1,4,7,10-tetraazacyclododecane-1,4,7,10-tetraacetic acid (DOTA), 1,4,7,10,13,16-hexaazacyclohexadecane-1,4,7,10,13,16-hexaacetic acid (HEHA), 2-(4-isothiocyanatobenzyl)-1,4,7,10,13,16-hexaazacyclohexadecane-1,4,7,10,13,16-hexaacetic acid (HEHA-NCS), [6,6′-({9-hydroxy-1,5-bis(methoxycarbonyl)-2,4-di(pyridin-2-yl)-3,7-diazabicyclo[3.3.1]nonane-3,7-diyl}bis(methylene))dipicolinic acid] (H₂Bispa²), N,N′-bis[(6-carboxy-2-pyridil)methyl]-4,13-diaza-18-crown-6 (Macropa), 6-[[16-[(6-carboxypyridin-2-yl)methyl]-1,4,10,13-tetraoxa-7,16-diazacyclooctadec-7-yl]methyl]-4-isothiocyanatopyridine-2-carboxylic acid (Macropa-NCS), Bis(phenyliminodiacetate)-diazacrown ether (Macropid) or t-Bu-calix[4]arene tetracarboxylic acid, preferably from DOTA or HEHA, more preferably from DOTA.
4. The alpha radiolabeled gastrin analogue of any one of items 1 to 3, wherein the alpha radionuclide is selected from ²¹²Bi, ²¹³Bi, ²²⁵Ac, ²²⁵Fm, ²¹¹At, ²²³Ra, ¹⁴⁹Tb, ²¹²Pb, ²²⁶Th and ²²⁷Th, preferably from ²¹²Bi, ²¹³Bi, ²²⁵Ac, ²²⁵Fm, ¹⁴⁹Tb, ²¹²Pb, ²²⁶Th and ²²⁷Th, and more preferably from ²¹³Bi, ²²⁵Ac and ¹⁴⁹Tb.
5. The alpha radiolabeled gastrin analogue of any one of items 1 to 4, wherein the alpha radionuclide is ²²⁵Ac.
6. The alpha radiolabeled gastrin analogue of any one of items 1 to 5, wherein the alpha radionuclide satisfies at least one of the following (i) and (ii):
   (i) a linear energy transfer (LET) of 20 keV/μm to 190 keV/μm, preferably of 30 keV/μm to 160 keV/μm, more preferably of 50 keV/μm to 150 keV/μm, in particular about 80 keV/μm; and
   (ii) a tissue penetration range (TPR) of 20 μm to 150 μm, preferably of 30 μm to 120 μm, more preferably of 40 μm to 100 μm.
7. The alpha radiolabeled gastrin analogue of any one of items 1 to 6, which is represented by the following formula (2):

(2)
(SEQ ID NO: 6)
Y-(DGlu)₆-Ala-Tyr-Gly-Trp-Nle-Asp-Phe-NH₂

wherein Y represents a moiety derived from DOTA that chelates ²²⁵Ac.
8. An alpha radiolabeled gastrin analogue for use in a method of treating one or more CCKB receptor positive diseases comprising the step of administering a therapeutically effective dose of an alpha radiolabeled gastrin analogue to a human subject diagnosed with said CCKB receptor positive disease(s);
   wherein the alpha radiolabeled gastrin analogue is as defined in any one of items 1 to 7.
9. The alpha radiolabeled gastrin analogue for the use of item 8, wherein the alpha radiolabeled gastrin analogue is represented by the following formula (2):

(2)
(SEQ ID NO: 6)
Y-(DGlu)₆-Ala-Tyr-Gly-Trp-Nle-Asp-Phe-NH₂

wherein Y represents a moiety derived from DOTA that chelates ²²⁵Ac.
10. The alpha radiolabeled gastrin analogue for the use of item 8 or 9, wherein the CCKB receptor positive disease(s) is/are selected from gastric cancer (GC), pancreatic adenocarcinoma (PADC), small-cell lung cancer (SCLC), extrapulmonary small-cell carcinoma (EPSCC), medullary thyroid cancer (MTC), gliomas, gastroenteropancreatic neuroendocrine tumors (GEP-NETs), colon cancer, ovarian cancer, breast cancer, and any CCKB receptor positive cancer or tumors.
11. The alpha radiolabeled gastrin analogue for the use of any one of items 8 to 10, wherein the CCKB receptor positive disease(s) is/are selected from SCLC, EPSCC and MTC, preferably from SCLC and MTC, and more preferably is MTC.
12. The alpha radiolabeled gastrin analogue for the use of any one of items 8 to 11, wherein the therapeutically effective dose that is administered to the human subject is of from 10 to 10,000 kBq/kg, preferably of from 30 to 1000 kBq/kg, more preferably of from 50 to 200 kBq/kg.
13. Method for treating CCKB receptor positive diseases comprising the administration of a therapeutically effective dose of an alpha radiolabeled gastrin analogue to a human subject in need thereof;
   wherein the alpha radiolabeled gastrin analogue is as defined in any one of items 1 to 7, and preferably as defined in item 7.

### BRIEF DESCRIPTION OF THE FIGURES

FIG. 1—Internalization of ¹⁷⁷Lu-PP-F11N and ²²⁵Ac-PP-F11N in A431/CCKBR cells. Internalized and cell-bound activity was measured after 2 h incubation with ¹⁷⁷Lu-PP-F11N or ²²⁵Ac-PP-F11N. All experiments were assayed in triplicate. Bars represent mean±SD.

FIG. 2—Biodistribution of ²²⁵Ac-PP-F11N in xenografted nude mice. Radioactivity uptake in tumors, kidneys, stomach, liver, bone and blood at 1, 4, 24, 48 h and 7 days after administration of ²²⁵Ac-PP-F11N shown as % of total injected radioactivity per gram of tissue (% i.A./g). Bars represent mean±SD, n=4 for each time point.

FIG. 3—Biodistribution of ²²⁵Ac-PP-F11N and ¹⁷⁷Lu-PP-F11N in xenografted nude mice. Comparative analysis of the radioactivity uptake in indicated organs at 4 h post injection of ²²⁵Ac-PP-F11N (black bars) and ¹⁷⁷Lu-PP-F11N (white bars). Bars represent mean±SD, n=4. *P<0.01, **P<0.01, ***P<0.001

FIG. 4—Tumor growth inhibition and prolonged life span in ²²⁵Ac-PP-F11N-treated mice. After tumor implantation, PBS or 30, 45, 60, 90 and 120 kBq of purified ²²⁵Ac-PP-F11N was administered into A431/CCKBR tumor-bearing nude mice groups as indicated (n=9 exempt from the control and 60 kBq group; n=18). Tumor growth curves in different treatment groups. Values are expressed as mean±SD.

FIG. 5—Histological analysis of kidney and stomach sections after treatment. Representative images of HE stains of organs isolated from control, 60 and 120 kBq ²²⁵Ac-PP-F11N-treated A431/CCKBR xenografted nude mice.

FIG. 6—SPECT/CT imaging after treatment with 45 kBq or 60 kBq of ²²⁵Ac-PP-F11N. SPECT-CT images with ¹¹¹In-PP-F11N of two mice with no detectable tumors after treatment with 45 kBq (left) and 60 kBq (right) of ²²⁵Ac-PP-F11N.

FIG. 7—Quality control of ²²⁵Ac-PP-F11N. Samples from fractions collected during HPLC purification were eluted on a thin-layer chromatography (TLC) plate (0.1M succinic acid in water/acetonitrile, 40/60, v/v), and elution was stopped. A phosphor screen (MultiSensitive; available from PerkinElmer) was exposed to the TLC plate 30 min after elution (exposition time: 2 min) and then scanned using a Cyclone Plus Storage Phosphor System (PerkinElmer) at 300 dpi resolution to measure the activity distribution. (A) Gamma emission mainly due to unbound radionuclides (²²¹Fr, ²¹³Bi and ²⁰⁹Tl) was observed. (B) A phosphor screen (MultiSensitive; PerkinElmer) was exposed to the same TLC plate 24 hours after elution (exposition time: 2 min) and then scanned to measure the activity distribution, which now shows only daughter radionuclides originating from α-decayed ²²⁵Ac.

### DETAILED DESCRIPTION OF THE INVENTION

### 1. Definitions

The expression “gastrin analogue” as used herein refers to a class of compounds (peptides) structurally related to the endogenous peptide hormone “gastrin”, which can bind to the CCKBR. In particular, the expression “gastrin analogue” defines compounds containing the C-terminal pentapeptide Gly-Trp-Dxx-Asp-Phe-NH₂, (SEQ ID NO: 2), wherein Dxx is an amino acid isosteric with methionine, analogous to the C-terminal amino acid sequence found in CCKBR-binding endogenous peptide hormones such as gastrin, cholecystokinin (CCK) and minigastrin (Leu-(Glu)₅-Glu-Glu-Glu-Glu-Ala-Tyr-Gly-Trp-Met-Asp-Phe-NH₂)(SEQ ID NO: 3). The gastrin analogue can be chemically modified for allowing the covalent attachment of a chelating moiety for radiometals such as 1,4,7,10-tetraazacyclododecane-1,4,7,10-tetraacetic acid (DOTA).

The term “amino acid” as used herein refers to a compound that contains or is derived from at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. This term encompasses both naturally occurring amino acids as well as synthetic amino acids that are not found in nature. Unless specified otherwise or dictated otherwise by the context, all connections between adjacent amino acid groups are formed by peptide (backbone amide) bonds. The peptides described herein are listed in the conventional amino- to carboxy-direction from left to right.

The expression “amino acid isosteric with methionine” (or “methionine bioisostere”) as used herein refers to a natural or unnatural amino acid having a shape and/or electronic properties similar to those of methionine. As a result, a compound in which a methionine residue is replaced by an amino acid isosteric with methionine shows the same properties, e.g. binding affinity, agonistic activity, etc., as the original compound containing methionine. Here, the term “isosteric” is meant to encompass all amino acids, which are essentially isosteric with methionine in that the backbone and/or side chain of said amino acids are so defined that they mimic, e.g. sterically, electronically, etc., methionine. Examples of amino acids isosteric with methionine include isoleucine (Ile), norleucine (Nle), homo-cysteine (homo-Cys), 2-amino-5-heptenoic acid, homo-norleucine (homo-Nle), 2-amino-4-methoxybutanoic acid, telluro-methionine (Te-Met), seleno-methionine (Se-Met), and phenylglycine (Phg). In some aspects, the expression “amino acid isosteric with methionine” refers to an amino acid residue which, if replacing methionine in the amino acid sequence of minigastrin, produces a gastrin analogue that retains at least 20%, preferably at least 50%, more preferably at least 80% of the pharmacological (agonistic) activity of minigastrin towards CCKBR. Pharmacological activity can be determined by measuring the intracellular increase of calcitonin level in gastrin analogue-stimulated cells as described by Bläker et al. (Regulatory Peptides 2004, 118, 111-117).

The expression “moiety that comprises an alpha radionuclide” (or “moiety that chelates or (covalently) bonds an alpha radionuclide”) as used herein refers to a moiety (chelating agent or ligand) that can either (i) donate electrons to an alpha radionuclide to form a coordination complex therewith, i.e. by forming at least one coordinate covalent bond (dipolar bond) therewith, or (ii) covalently bonds an alpha radionuclide such as ²¹¹At and ²²³Ra. The chelating mechanism depends on the chelating agent and/or radionuclide. For example, it is believed that DOTA can coordinate a radionuclide, e.g. ²²⁵Ac, via carboxylate and amino groups (donor groups) thereby forming complexes having high stability (Dai et al. Nature Com. 2018, 9, 857). Non-limiting examples of moieties that can chelate an alpha radionuclide such as ²²⁵Ac include DOTA, 1,4,7,10,13,16-hexaazacyclohexadecane-1,4,7,10,13,16-hexaacetic acid (HEHA), 2-(4-isothiocyanatobenzyl)-1,4,7,10,13,16-hexaazacyclohexadecane-1,4,7,10,13,16-hexaacetic acid (HEHA-NCS), [6,6′-({9-hydroxy-1,5-bis(methoxycarbonyl)-2,4-di(pyridin-2-yl)-3,7-diazabicyclo[3.3.1]nonane-3,7-diyl}bis(methylene))dipicolinic acid] (H₂Bispa²), N,N′-bis[(6-carboxy-2-pyridil)methyl]-4,13-diaza-18-crown-6 (Macropa), 6-[[16-[(6-carboxypyridin-2-yl)methyl]-1,4,10,13-tetraoxa-7,16-diazacyclooctadec-7-yl]methyl]-4-isothiocyanatopyridine-2-carboxylic acid (Macropa-NCS), Bis(phenyliminodiacetate)-diazacrown ether (Macropid), t-Bu-calix[4]arene tetracarboxylic acid.

The expression “moiety derived from a compound” as used herein refers to a moiety (Y) bonded to an adjacent moiety, which differs from the molecule from which it is derived only by the structural element(s) responsible for bonding to the adjacent moiety. This may include covalent bonds formed by existing functional groups or covalent bonds and adjacent functional groups newly introduced for this purpose. For instance, the expression “moiety derived from 1,4,7,10-tetraazacyclododecane-1,4,7,10-tetraacetic acid (DOTA)” refers to a DOTA molecule that has been covalently attached to the N-terminus of the peptide *-(DGlu)₆-Ala-Tyr-Gly-Trp-Axx-Asp-Phe-NH₂ (SEQ ID NO: 7) via one of its carboxyl groups to form a peptide (amide) linkage (* indicates covalent attachment to the DOTA moiety).

The expression “alpha radionuclide” (or “alpha particle-emitting nuclide”, “alpha emitter”, “alpha”) in this connection refers to an unstable element (radionuclide) that undergoes radioactive decay by emitting alpha particles. Non-limiting examples of alpha radionuclides include ²¹²Bi, ²¹³Bi, ²²⁵Ac, ²²⁵Fm, ²¹¹At, ²²³Ra, ¹⁴⁹Tb, ²¹²Pb, ²²⁶Th and ²²⁷Th.

The term “cancer” as used herein means the pathological condition in mammalian tissues that is characterized by abnormal cell growth to form malignant tumors, which may have the potential to invade or spread to other tissues or parts of the body to form “secondary” tumors known as metastases. A tumor comprises one or more cancer cells.

The term “internalization” as used herein refers to the biological process in which molecules, e.g. an alpha radiolabeled gastrin analogue, are engulfed by the cell membrane and drawn into the cell. As a result, the molecules, e.g. the alpha radiolabeled gastrin analogue, are present inside the cell.

The expression “cell uptake” refers to the biological process in which molecules are internalized and/or bound on the cell membrane. As a result, the molecules can be present inside the cell as well as at the cell membrane. In an analogous manner, the expression “tumor uptake” (or “tumor cell uptake”) refers to the biological process in which molecules, e.g. a radiolabeled gastrin analogue, are taken up by tumor (cancer) cells. As a result, the molecules, e.g. the radiolabeled gastrin analogue, can be present inside the tumor (cancer) cell and/or at the cell membrane.

The expression “CCKB receptor positive diseases” as used herein refer to diseases, e.g. tumors or cancers, which are characterized by (over-)expression of the CCKBR on the cell surface (Reubi et al. Cancer Res. 1997, 57(7), 1377-1386; Dufresne et al. Physiol Rev 2006, 86, 805-847). The (over-)expression of CCKBR, particularly on the cell surface, can be determined by techniques known in the art, e.g. by immunohistochemistry or Western Blot analysis as described further below. Non-limiting examples of CCKBR positive diseases include gastric (stomach) cancer (GC), pancreatic adenocarcinoma (PADC), small-cell lung cancer (SCLC), extrapulmonary small-cell carcinoma (EPSCC), medullary thyroid cancer (MTC), gliomas, gastroenteropancreatic neuroendocrine tumors (GEP-NETs), colon cancer, ovarian cancer, breast cancer. In the context of the present disclosure, the expression “CCKB receptor positive diseases” is also meant to encompass diseases resulting from the metastatic spread of a primary tumor to another part of the body, i.e. advanced stage diseases. For instance, if a CCKB receptor disease (cancer) arises in the colon and metastasizes to another part of the body, the cancer cells found in this other part of the body are (CCKBR positive) colon cancer cells.

The expression “human subject diagnosed with a CCKBR positive disease” as used herein refers to a human subject having a positive diagnosis with respect to at least one of the aforementioned CCKBR positive diseases, for instance a positive diagnosis of MTC or SCLC. In this connection, the diagnosis and state of disease(s) can be determined by a physician based on established screening guidelines, e.g. as available from the American Cancer Society (ACS). For instance, a “positive diagnosis” can mean that the subject has a histological and/or cytological status of disease and, optionally, one or more of the following:
(1) radiographically documented disease progression or recurrence after at least one systemic treatment regimen,
(2) at least one non-irradiated extracranial measurable target lesion as per the Response Evaluation Criteria in Solid Tumors 1.1 (RECIST 1.1) documented after the last anticancer therapy,
(3) at least one tumor lesion as per Positron Emission Tomography Response Criteria in Solid Tumors 1.0 (PERCIST 1.0), and
(4) an Eastern Cooperative Oncology Group (ECOG) score of 0-2.

The expression “effective dose” (or “effective amount”) as used herein refers to the total amount of radioactivity (in Becquerels/kilogram) administered to a subject (patient) in order to perform treatment of the disease(s), e.g. the dose required for reducing or stopping cancer cell proliferation and/or reducing the number of proliferating cancer cells. If the course of treatment includes one or more (administration) cycles, the effective dose refers to the total amount of radioactivity administered to a subject over the entire course, i.e. over all cycles. The term “cycle” in this connection refers to a period of time wherein the compound is administered to the subject (treatment time) and then the patient is allowed to rest (rest time) before entering another cycle. The treatment can include one or more cycles, e.g. up to ten cycles. A series of cycles is usually called a “course”, which can last over several months, e.g. 3 to 6 months, depending on the length of each cycle.

The effective dose can be determined by a physician based on dosimetry. The effective dose and frequency of dosage for any particular subject/patient can vary and depends on a variety of factors including the patient's age, body weight, general health, sex, diet, mode and time of administration, rate of excretion, the severity of the disease, and the individual undergoing therapy. These factors are considered by the physician when determining the effective dose.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless the context dictates otherwise and/or alternative meanings are explicitly provided herein, all terms are intended to have meanings generally accepted in the art, as reflected by IUPAC Gold Book (status of 1^{st} Nov. 2017), or the Dictionary of Chemistry, Oxford, 6^{th} Ed.

### 2. Overview

The present invention is based on the discovery that the labeling of a specific gastrin analogue (i.e. a gastrin analogue of formula (1)) with an alpha radionuclide, in particular with ²²⁵Ac, leads to excellent uptake into target cells, e.g. cancer cells, resulting in excellent biodistribution and therapeutic efficacy, while toxicity (adverse side effects) for healthy tissues, i.e. for the tissues endogenously expressing CCKBR and/or tissues surrounding the target cells, are prevented and/or reduced. These results are particularly surprising because the use of alpha radiolabeled compounds has been associated with cytotoxic side effects, e.g. radiation nephropathy, hemorrhagic gastritis, etc., likely due to the high energy of the emitted alpha particles.

The compound of the present invention can be used in methods of treating CCKBR positive diseases, in particular GC, PADC, SCLC, EPSCC, MTC, gliomas, GEP-NETs, colon cancer, ovarian cancer, breast cancer, and any CCKB receptor positive diseases. Moreover, it is expected that the compound of the present invention is particular suited for treating CCKBR positive diseases having reached an advanced (metastatic) stage of disease, for instance metastatic MTC or metastatic SCLC.

### 3. Alpha Radiolabeled Gastrin Analogue

The present invention relates to a gastrin analogue (compound) labeled with an alpha radionuclide, i.e. ²²⁵Ac. The compound of the present invention exhibits excellent in vivo biodistribution and therapeutic efficacy towards CCKBR-expressing target cells, e.g. cancer cells. Furthermore, the compound of the present invention exhibits low toxicity (or no toxicity) for tissues endogenously expressing CCKBR and/or tissues surrounding the target cells.

Without being bound to any theory, it is believed that the use of a gastrin analogue of formula (1) allows to achieve excellent targeting and uptake effects in CCKBR-expressing cells resulting in excellent biodistribution while labeling with an alpha radionuclide allows to achieve excellent therapeutic efficacy (i.e. a high level of DNA damage in target cells) due to the high energy of the emitted alpha particles. Furthermore, the present inventors have observed that, surprisingly, the use of an alpha radiolabeled gastrin analogue does not lead to adverse side effects (toxicity) in the healthy tissues endogenously expressing CCKBR (e.g. in the stomach) and/or surrounding the target cells. These results are presumably due to the fact that the compound of the present invention shows a low level of accumulation in healthy tissues, and also that unfavorable radiation of the alpha radionuclide to surrounding tissues is significantly reduced due to its short penetration range (i.e. reduced cross-fire effect). This is in contrast with other radionuclides such as ¹⁷⁷Lu which may cause damage to surrounding healthy tissues due to their long penetration range (cross-fire effect).

The alpha radiolabeled gastrin analogue of the present invention is a compound represented by the following formula (1):

Y-(DGlu)₆-Ala-Tyr-Gly-Trp-Axx-Asp-Phe-NH₂(SEQ ID NO: 5) (1)
wherein Axx represents an amino acid isosteric with methionine, and Y represents a moiety that chelates an alpha radionuclide.

In one embodiment, Axx represents an amino acid isosteric with methionine selected from the group consisting of isoleucine (Ile), norleucine (Nle), 2-amino-5-heptenoic acid, homo-norleucine (homo-Nle), homo-cysteine (homo-Cys), 2-amino-4-methoxybutanoic acid, telluromethionine (Te-Met), selenomethionine (Se-Met) and phenylglycine (Phg). Preferably, Axx is Nle.

In one embodiment, Y represents a moiety derived from a chelating agent selected from 1,4,7,10-tetraazacyclododecane-1,4,7,10-tetraacetic acid (DOTA), 1,4,7,10,13,16-hexaazacyclohexadecane-1,4,7,10,13,16-hexaacetic acid (HEHA), 2-(4-isothiocyanatobenzyl)-1,4,7,10,13,16-hexaazacyclohexadecane-1,4,7,10,13,16-hexaacetic acid (HEHA-NCS), [6,6′-({9-hydroxy-1,5-bis(methoxycarbonyl)-2,4-di(pyridin-2-yl)-3,7-diazabicyclo[3.3.1]nonane-3,7-diyl}bis(methylene))dipicolinic acid](H₂Bispa²), N,N′-bis[(6-carboxy-2-pyridil)methyl]-4,13-diaza-18-crown-6 (Macropa), 6-[[16-[(6-carboxypyridin-2-yl)methyl]-1,4,10,13-tetraoxa-7,16-diazacyclooctadec-7-yl]methyl]-4-isothiocyanatopyridine-2-carboxylic acid (Macropa-NCS), Bis(phenyliminodiacetate)-diazacrown ether (Macropid) or t-Bu-calix[4]arene tetracarboxylic acid. Preferably, Y represents a moiety derived from DOTA or HEHA.

More preferably, Y is DOTA.

In a preferred embodiment, Axx is Nle and Y is DOTA. In the following, this compound is designated as “PP-F11N”.

In one embodiment, the alpha radionuclide is selected from ²¹²Bi, ²¹³Bi, ²²⁵Ac, ²²⁵Fm, ²¹¹At, ²²³Ra, ¹⁴⁹Tb, ²¹²Pb, ²²⁶Th and ²²⁷Th. Preferably, the alpha radionuclide is selected from ²¹²Bi, ²¹³Bi, ²²⁵Ac, ²²⁵Fm, ¹⁴⁹Tb, ²¹²Pb, ²²⁶Th and ²²⁷Th. More preferably, the alpha radionuclide is selected from ²¹³Bi, ²²⁵Ac and ¹⁴⁹Tb, and most preferably the alpha radionuclide is ²²⁵Ac.

In one embodiment, the alpha radionuclide satisfies at least one, and preferably both, of the following (i) and (ii):
(i) a linear energy transfer (LET) of 20 keV/μm to 190 keV/μm, preferably of 30 keV/μm to 160 keV/μm, more preferably of 50 keV/μm to 150 keV/μm, in particular about 80 keV/μm; and
(ii) a tissue penetration range (TPR) of 20 μm to 150 μm, preferably of 30 μm to 120 μm, more preferably of 40 μm to 100 μm.

In a more preferred embodiment, the alpha radiolabeled gastrin analogue is represented by the following formula (2):

(2)
(SEQ ID NO: 6)
Y-(DGlu)₆-Ala-Tyr-Gly-Trp-Nle-Asp-Phe-NH₂

wherein Y represents a moiety derived from DOTA that chelates ²²⁵Ac. In the following, this compound is designated as “²²⁵Ac-PP-F11N”.

The alpha radiolabeled gastrin analogue can be provided in the form of a pharmaceutical composition for usage in human medicine. Such composition typically comprises a therapeutically effective dose of an alpha radiolabeled gastrin analogue of the invention and one or more other components, e.g. a carrier, a diluent, etc. In one aspect, the pharmaceutical composition can comprise a mTOR inhibitor, in particular rapamycin and/or a rapalog. In another aspect, the pharmaceutical composition is free of mTOR inhibitor, in particular free of rapamycin and/or a rapalog. The term “mTOR inhibitor” in this connection refers to a compound which inhibits the mammalian target of rapamycin (mTOR). The term “rapamycin” (Sirolimus) refers to a macrolide compound, which is known in the art to exhibit immunosuppressant properties by inhibiting the mammalian target of rapamycin (mTOR), whereas the term “rapalog” (which stands for “rapamycin-analog”) refers to a class of compounds structurally related to rapamycin, which are known to inhibit the mammalian target of rapamycin in complex 1 (mTORC1) by binding to the FK-binding protein 12. Examples of rapalogs include Everolimus (RAD001), Temserolimus (CCI-779) and Ridaforolimus (AP-23573, MK-8669).

### 4. Use of Compound in Methods of Treating Diseases

The compound of the present invention can be used in methods of treating one or more CCKBR positive diseases. The treatment can be a therapeutic and/or a prophylactic treatment, with the aim being to prevent, reduce or stop the progression of the CCKBR positive disease(s) via targeted destruction of the tumor cells. In some aspects, the treatment can prolong survival of a patient as compared to expected survival if not receiving the treatment.

The method of treating the CCKBR positive disease(s) comprises the step of administering a therapeutically effective dose of an alpha radiolabeled gastrin analogue (or a pharmaceutical composition comprising the same) to a human subject diagnosed with one or more CCKBR positive diseases. The alpha radiolabeled gastrin analogue to be administered to the subject is a compound represented by the formula (1) described above. Preferably, the compound to be administered to the subject is PP-F11N labeled with an alpha radionuclide.

In one preferred embodiment, the alpha radiolabeled gastrin analogue to be administered to the subject is ²²⁵Ac-PP-F11N.

The CCKB receptor disease(s) to be treated is not particularly limited provided that the target cells are characterized by the expression of CCKBR (CCKBR positive). In one embodiment, the CCKB receptor positive disease(s) to be treated is/are selected from gastric (stomach) cancer (GC), pancreatic adenocarcinoma (PADC), small-cell lung cancer (SCLC), extrapulmonary small-cell carcinoma (EPSCC), medullary thyroid cancer (MTC), gliomas (e.g. astrocytomas), gastroenteropancreatic neuroendocrine tumors (GEP-NETs), colon cancer, ovarian cancer, breast cancer, and any CCKB receptor positive cancer or tumors.

In one embodiment, the CCKB receptor positive disease(s) to be treated is/are selected from SCLC, EPSCC and MTC. Preferably, the CCKB receptor positive disease(s) to be treated is/are SCLC and/or MTC. More preferably, the CCKB receptor positive disease is MTC.

Furthermore, it is expected that the compound of the present invention, e.g. ²²⁵Ac-PP-F11N, exhibits superior therapeutic efficacy when administered to a subject diagnosed with at least one of the aforementioned diseases that has/have reached an advanced (metastatic) stage, because the emitted alpha particles can lead to a high level of DNA double strand break in disseminated cancer cells. This is contrast with other radionuclides such as lutetium-177 which may insufficient efficacy for treating disseminated diseases, presumably due to the activation of DNA-repair mechanisms and radioresistance. Accordingly, the disease(s) to be treated can be any CCKB receptor positive disease at an advanced stage, preferably one or more selected from the aforementioned CCKB receptor positive diseases at an advanced stage, in particular metastatic MTC or metastatic SCLC.

The therapeutic effect that is observed can be a reduction in the number of cancer cells, a reduction in tumor size, an inhibition or retardation of cancer cell infiltration into peripheral organs, an inhibition of tumor growth, and/or a relief of one or more of the symptoms associated with the CCKBR positive disease(s).

The therapeutically effective dose can be determined by a physician on a routine basis. The dose level and frequency of dosage for any particular subject/patient can vary and depends on a variety of factors including the activity of the compound employed, the metabolic stability and length of action of that compound, the patient's age, body weight, general health, sex, diet, mode and time of administration, rate of excretion, drug combination, the severity of the particular condition, and the individual undergoing therapy. These factors are considered by the physician when determining the therapeutically effective dose.

In one embodiment, the therapeutically effective dose of the alpha radiolabeled gastrin analogue that is administered to the patient is of from 10 to 40,000 kBq/kg. Preferably, the therapeutically effective dose is of from 30 to 1,000 kBq/kg, more preferably of from 50 to 200 kBq/kg. The alpha radiolabeled gastrin analogue can be administered once or several times daily.

The molecule can be administered to the patient at one time or over a series of treatments, i.e. over one or more administration cycles. In particular, the compound can be administered to the subject once or twice per cycle of two to ten weeks, preferably once per cycle of four to eight weeks, more preferably once per cycle of six weeks or once per cycle of eight weeks. When the compound is administered twice per cycle, the therapeutically effective dose is split in two half-doses which are administered separately over the course of the cycle. The number of cycles can range from one to a maximum of ten cycles, preferably two to eight cycles, more preferably four to six cycles.

In one aspect, the compound is administered to the subject according to one of the following administration patterns:
(A) once per cycle of six weeks for at least two six-week cycles, preferably once per cycle of six weeks for three six-week cycles; or
(B) once per cycle of eight weeks for at least two eight-week cycles, preferably once per cycle of eight weeks for three eight-week cycles.

In one embodiment, the patient diagnosed with one or more CCKBR positive diseases satisfies one or more of the following (a) to (h) prior to the administration of the compound:
(a) a serum creatinine of less than or equal to 1.5×ULN or an estimated glomerular filtration rate (eGFR) greater than 50 mL/min based on the Chronic Kidney Disease-Epidemiology Collaboration (CKD-EPI) equation;
(b) a blood absolute neutrophil count (ANC) equal to or greater than 1,500 cells/μL;
(c) a blood platelet count equal to or greater than 100,000 cells/μL;
(d) a blood haemoglobin equal to or greater than 9 g/dL;
(e) AST or ALT equal to or less than 3×ULN;
(f) a serum albumin greater than 20 g/L;
(g) a total bilirubin equal to or less than 2.5 mg/dL; and
(h) an alkaline phosphatase less than 5×ULN.

In one embodiment, the method can include a step of determining whether a subject meets one or more of the (a) to (h) above.

In one embodiment, the compound is administered to the subject by injection, in particular by intravenous injection. In this connection, the compound can be provided as a solution in a pharmaceutically acceptable injectable carrier such as an aqueous carrier (e.g. water or 0.9% sodium chloride). The effective dose can be administered in a volume of 1 to 200 mL, preferably 5 to 50 mL, such as about 10 mL. The infusion rate can be of from 35 to 60 mL/h, for instance about 50 mL/h.

According to one embodiment, the method comprises the steps of:
(α) preparing an injectable solution of the compound by dissolving the compound in a pharmaceutically acceptable injectable carrier to obtain an injectable solution of the compound; and
(β) administering the injectable aqueous solution of the compound obtained from step (α) to the patient, preferably at an infusion rate of 35 to 60 mL/h such as 50 mL/h over an infusion period of 20 to 60 min, e.g. 30 to 45 min.

According to one further embodiment, the compound is administered concurrently with, before and/or after one or more other therapeutic agents or therapies such as chemotherapeutic agents, immunomodulatory agents, proton pump inhibitors (PPIs) or histamine H₂-receptor antagonists.

In one aspect, the compound (or pharmaceutical composition comprising the same) is administered in combination with (i.e. concurrently with, before and/or after) rapamycin and/or a rapalog. In one further aspect, the compound is administered in combination (i.e. concurrently with, before and/or after) with Everolimus. In yet another aspect, the compound is not administered in combination with (i.e. concurrently with, before and/or after) rapamycin and/or a rapalog.

In one embodiment, the compound of the present invention can be administered concurrently with, before or after one or more other therapeutic agents or therapies such as chemotherapeutic agents and/or immunomodulatory agents. Examples of therapeutic agents or therapies that can be used include antineoplastic agents such as alkylating agents, alkaloids or kinase inhibitors, immunomodulatory agents and pharmaceutically acceptable salts and derivatives thereof.

### 5. Preparation of the Alpha Radiolabeled Gastrin Analogue

In the following, methods are provided for the preparation of the radiolabeled gastrin analogue. The gastrin analogue can be synthesized relying on standard Fmoc-based solid-phase peptide synthesis (SPPS), including on-resin peptide coupling and convergent strategies. The general strategies and methodology which can be used for preparing and radiolabeling the gastrin analogue of the present invention are well-known to the skilled person and also described further below.

In some aspects, the present disclosure provides a method for purifying and determining the purity of a compound labeled with an alpha radionuclide such as ²²⁵Ac. The purification and quality control of alpha radiolabeled compounds, e.g. ²²⁵Ac-labeled compounds, may be particularly difficult because alpha radionuclides are short-range emitters usually showing α-decay but, in some instances, no γ-emission. Accordingly, compounds labeled with alpha radionuclides may not be (directly) detectable by conventional radiographic systems. Furthermore, it may be impossible to detect alpha radiolabeled compounds by standard detection methods, for instance by UV absorbance, due to their low concentration in the purification solution (typically in the picomolar range).

The present inventors have therefore developed a method for purifying and determining the purity of alpha radiolabeled compounds. This method is described below using DOTA-(DGlu)₆-Ala-Tyr-Gly-Trp-Nle-Asp-Phe-NH₂ (SEQ ID NO: 4) labeled with ²²⁵Ac (²²⁵Ac-PP-F11N) as an exemplary compound.

(1) A compound comprising a chelating moiety (e.g. PP-F11N) is labeled with ²²⁵Ac (e.g. by reacting the compound with ²²⁵Ac in solution as described further below) and then the labeling solution is purified by a chromatographic method (HPLC), wherein fractions of the eluted solution are collected;

(2) The fractions are sampled and individually developed (eluted) on a solid support comprising a stationary phase (e.g. on a TLC plate) using an appropriate solvent system (e.g. 0.1 M succinic acid in water/acetonitrile, 40/60, v/v) and, thereafter, allowed to stand for a predetermined period of time (e.g. for a period of 6 to 24 hours) until (e.g. unbound) γ-ray emitting radionuclides present in the labeling solution are decayed, and only daughter radionuclides originating from ²²⁵Ac (produced by α-decay of ²²⁵Ac) are present;

(3) The activity distribution of the plate is measured and the purity of the ²²⁵Ac-labeled compound in the fraction can be determined.

The activity distribution on the TLC can be measured by using a Cyclone® Plus Storage Phosphor System and MultiSensitive or Super Resolution storage phosphor screens (available from PerkinElmer, Switzerland), which may be appropriately calibrated. To conduct the measurement, a phosphor screen (preliminary erased using a white light box) is exposed to the TLC plate, placed in the Cyclone® Plus radioimaging system and scanned. The exposition time (phosphor screen→TLC plate) may be from 1 to 60 min. preferably from 2 to 10 min, more preferably 2 min. The determination of purity/quantitation of the compound in the collected fraction can be performed using OptiQuant® software.

The method described above enables the detection and quantification of alpha radiolabeled compounds with high sensitivity.

### 6. Examples

### 6.1 List of Abbreviations

DIEA: diisopropylethylamine

DMEM: Dulbecco's Modified Eagle Medium

DMF: dimethyl formamide

DTT: dithiothreitol

ESI: electron spray ionization

FBS: fetal bovine serum

HATU: 1-[Bis(dimethylamino)methylene]-1H-1,2,3-triazolo[4,5-b]pyridinium 3-oxide hexafluorophosphate

HBTU: 3-[Bis(dimethylamino)methyliumyl]-3H-benzotriazol-1-oxide hexafluorophosphate

HPLC: high-performance liquid chromatography

IU: international unit

MS: mass spectrometry

PBS: phosphate-buffered saline

SQD: single quadrupole detection

SPECT: single-photon emission computed tomography

SPPS: solid-phase peptide synthesis

TBST: tris-buffered saline with Tween 20

TFA: trifluoroacetic acid

TIS: triisopropylsilane

TLC: thin layer chromatography

UPLC: ultra-performance liquid chromatography

### 6.2 Materials and Methods

The following materials and methods can be used to prepare and evaluate the compound of the present invention.

### 6.2.1 Cell Culture, Transfection and Treatments

Human epidermoid carcinoma A431 stable cell line that overexpress CCKBR, generated as previously described (Aloj et al., J Nucl Med 2004, 45(3), 485-94) can be cultured in DMEM, supplemented with 10% FBS (Bio Concept, Switzerland), 2 mM glutamine and antibiotics (0.1 mg/mL streptomycin, 100 IU penicillin) at 37° C. in a humidified incubator containing 5% CO₂.

### 6.2.2 Preparation of Gastrin Analogues

The gastrin analogues described herein can be prepared by standard Fmoc-based SPPS, including on-resin peptide coupling and convergent strategies using an Activo-P-11 Automated Peptide Synthesizer (Activotec) and a Rink Amide resin (loading: 0.60 mmol/g; Novabiochem).

Coupling reactions for amide bond formation are performed over 30 min at room temperature using 3 eq of Fmoc-amino-acids activated with HBTU (2.9 eq) in the presence of DIEA (6 eq.). Fmoc deprotection is conducted with a solution of 20% piperidine in DMF. Coupling of the N-terminal labeling moiety is performed over 30 min at room temperature using 3 eq of DOTA tris-t-Bu ester (Novabiochem) activated with HATU (2.9 eq) in the presence of DIEA (6 eq).

The peptides are cleaved from the resin under simultaneous side-chain deprotection by treatment with TFA/TIS/water (95/2.5/2.5, v/v/v) during 60 min. After concentration of the cleavage mixture, the crude peptides are precipitated with cold diethyl ether and centrifugated.

The peptides can be purified on a Waters Autopurification HPLC system coupled to SQD mass spectrometer with a XSelect Peptide CSH C18 OBD Prep column (130 Å, 5 μm, 19 mm×150 mm) using solvent system (0.1% TFA in water) and B (0.1% TFA in acetonitrile) at a flow rate of 25 mL/min and a 20-60% gradient of B over 30 min. The appropriate fractions are associated, concentrated and lyophilized. The purity is determined on a Waters Acquity UPLC System coupled to SQD mass spectrometer with CSH C18 column (130 Å, 1.7 μm, 2.1 mm×50 mm) using solvent system A (0.1% TFA in water) and (0.1% TFA in acetonitrile) at a flow rate of 0.6 mL/min and a 5-85% gradient of B over 5 min.

MS-analysis can be performed using electrospray ionization (ESI) interface in positive and negative mode.

### 6.2.3 Radiolabeling of Gastrin Analogues

(a) To prepare a gastrin analogue radiolabeled with an alpha radionuclide, i.e. with actinium-225, a stock solution of N-terminally DOTA-conjugated gastrin analogue DOTA-(DGlu)₆-Ala-Tyr-Gly-Trp-Nle-Asp-Phe-NH₂ (SEQ ID NO: 4) (PP-F11N; prepared as described in section 6.2.1 above) in metal free water is prepared and stored at −20° C. (concentration: 0.58 mM).

A reaction mixture comprising 6 μL of ²²⁵Ac solution (6 MBq in 0.1 M HCl; available from ITG GmbH, Germany), 100 μL of PP-F11N stock solution (0.58 mmol), 120 μL ammonium acetate (0.4 M, pH 5.5) and 21 μL ascorbic acid (100 mg/mL freshly prepared in sodium acetate buffer; total pH=5-5.5) is heated for 15 min at 90° C. in a clean and sterile Eppendorf® tube (1.5 mL). Then, 2 μL of 0.5 mM EDTA in metal free water is added in order to complex any free metals in the reaction mixture.

The reaction batch can be purified using a Merck Hitachi LaChrom 2D HPLC system, equipped with an autosampler, a radiation monitor (RM-19, EBERLINE Instrument Corporation, SANTA FE, New Mexico), a UV detector (Pharmacia LKB-UV-M II), a 515 HPLC pump and a L-7100 pump connected with a reversed-phase C-18 Stability BS-C23 column from Dr. Maisch (150×4.6 mm) (5 μm, 150×4.6 mm, Xterra™, MS, Waters, USA). The mobile phase consists of 0.1% TFA (Sigma-Aldrich, USA) in metal free water (A) and acetonitrile (VWR Chemicals, USA; HPLC-grade) (B). After injection of sample, solvent A (68%) in solvent B at a flow rate of 3 mL/min is applied for 5 min to load all sample onto the column. Then, a gradient of solvent A (50-10%) in solvent B at a flow rate of 1 mL/min is applied for 25 min to separate ²²⁵Ac-PP-F11N from unlabeled PP-F11N. The elution time of ²²⁵Ac-labeled PP-F11N is higher than unlabeled PP-F11N, after the UV peak of unlabeled PP-F11N. Clean and sterile Eppendorf© tubes with 250 μL 100 mg/mL fresh sodium acetate buffer can be used to collect ²²⁵Ac-PP-F11N fractions.

The purity of the obtained ²²⁵Ac-PP-F11N fractions can be determined by developing a sample thereof on a TLC plate (TLC plate coated with RP-18 modified silica gel 60, 5×10 cm; Merck) using 0.1 M succinic acid in water/acetonitrile 40/60 (v/v) as an eluent. The TLC plate is allowed to stand for 24 hours and, thereafter, placed in a cassette contained a MultiSensitive storage phosphor screen (available from PerkinElmer). After 2 minutes, the phosphor screen is placed in a Cyclone® Plus Storage Phosphor System and the activity distribution is measured.

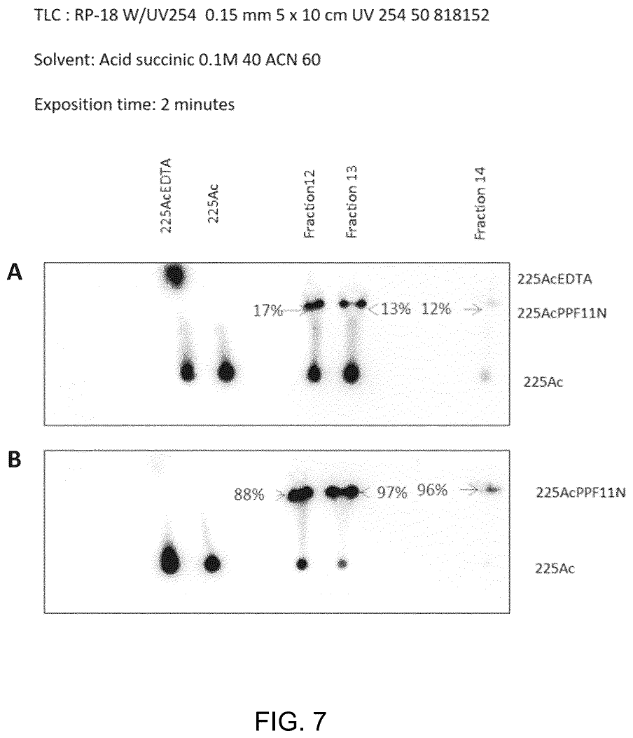

The purity of the ²²⁵Ac-PP-F11N compound in the collected fraction used in the following experiments was determined to be 97% (FIG. 7).

(b) To prepare a gastrin analogue radiolabeled with indium-111 (used for SPECT imaging after ²²⁵Ac-PP-F11N-treatment), 23.4 nmol of PP-F11N and 120 MBq of ¹¹¹In (69.5 pmol, available from ITG GmbH, Germany) are prepared in 92 μL 0.4 M ammonium acetate buffer (pH 5.5), 66 μL 0.5 M ascorbic acid is added and labeling is carried out at 95° C. for 20 min. Then, 2 μL of 0.5 mM EDTA in metal free water is added in order to complex any free metals in the reaction mixture.

The indium incorporation can be analyzed by standard HPLC using a C18 column and reached above 95% efficiency. Directly after labeling, a gamma counter is used to prepare appropriate dilutions of radiolabeled gastrin analogues for targeted radiation experiments.

(c) To prepare a gastrin analogue radiolabeled with lutetium-177 (used for comparative experiments), a solution of PP-F11N and ¹⁷⁷Lu (available from ITG GmbH, Germany) in a nuclide/peptide ratio of 1:30 is prepared in 0.4 M ammonium acetate buffer (pH 5.5) and labeling is carried out at 90° C. for 15 min.

The lutetium incorporation can be analyzed by standard HPLC using a C18 column and reached above 95% efficiency. Directly after labeling, a gamma counter is used to prepare appropriate dilutions of radiolabeled gastrin analogues for targeted radiation experiments.

### 6.2.4 In Vitro Radiolabelled Peptide Internalization Assays

For the internalization assay, 1×10⁶ cells per well are cultivated on 6-well plates overnight. On the next day, PBS-washed cells are incubated with 160 Bq of purified ²²⁵Ac-PP-F11N (or 100.000 cpm of ¹⁷⁷Lu-PP-F11N) in DMEM with 0.1% BSA at standard tissue culture condition for two hours. 4 μM LEEEEEAYGWMDF (SEQ ID NO: 1) peptide is used for blocking experiments.

After incubation, the supernatant (together with 2×PBS wash solutions) is collected. Then, the cells are incubated twice in 0.05 M ice-cold glycine buffer (pH=2) for 5 min followed by a dissolving step in 1 M NaOH for 15 min at 37° C. All three collected fractions (supernatant/PBS; glycine solution; dissolved cells) can be measured on a Packard Cobra II Auto-Gamma counter (PerkinElmer, Switzerland). Internalized and membrane-bound fractions of ²²⁵Ac-PP-F11N are shown as % of total activity. Unspecific membrane binding (glycine fraction) or internalization (NaOH-dissolved cells) from the experiments with blocking peptides are subtracted from the obtained results.

### 6.2.5 Proliferation Assay

For the cell proliferation assay, 4×10³ cells per well are seeded on the 96-well plates. Next day, different radioactivity levels of ²²⁵Ac-PP-F11N (0.01 to 316.23 kBq/mL) are added to the A431/CCKBR cells. After 2 h incubation, medium containing unbound ²²⁵Ac-PP-F11N is removed and the cells are incubated for another 24 h in fresh medium.

Cell proliferation can be analyzed using CellTiter 96 AQueous Non-Radioactive Cell Proliferation Kit (Promega AG, Switzerland) according to the manufacturer's instruction. Absorbance of formazan product is measured at 570 nm with a reference of 650 nm using a MicroPlate Reader (PerkinElmer, Switzerland). The absorbance of the control (untreated) cells is set as 100% cell viability. The activity level of ²²⁵Ac-PP-F11N resulting in 50% cell viability is calculated and presented as the half-maximal effective activity level (EA₅₀) in cell-killing. The assay is performed in triplicate.

### 6.2.6 WB Analysis

The expression levels of CCKBR can be determined by Western Blot analysis as follows:

Antibodies against CCKBR (ab77077) are available from Abcam (United Kingdom). Cells are homogenized in lysis buffer (50 mM Tris-HCl pH 7.5, 150 mM NaCl, 1% Triton X, 0.1% SDS supplemented with 1 mM sodium orthovanadate, 1 mM NaF and protease inhibitor cocktail (Roche)). Aliquots of 50 μg protein extracts are separated by SDS-PAGE and transferred to PVDF membranes (Millipore) by electroblotting. Membranes are blocked with 5% skim milk in TBST (0.1% Tween 20) for 1 h and incubated with 2% BSA in TBST overnight with the primary antibody followed by 2 h incubation with HRP-conjugated secondary antibody. Protein-specific signals can be detected by a chemiluminescence reagent (ECL) and signals can be acquired by using ImageQuant RT ECL Imager (GE Healthcare).

Prior to CCKBR detection, protein lysates are subjected to deglycosylation. Briefly, 18 μL of whole cell lysate (approx. 50 μg) are mixed with 2 μL of 10× denaturing buffer (5% SDS, 0.4 M DTT) and incubated for 10 min at RT. Next, 4 μL of 10× Glycobuffer (0.5 M sodium phosphate, pH 7.5), 4 μL of 10% Tween-20 and 10 μL of water were added. Finally, 2 μL of PNGase F (Sigma) is added, mixed and collected by centrifugation. The reaction is carried out at 37° C. overnight before WB analysis.

### 6.2.7 Animal Study

In the study human A431/CCKBR xenograft mouse model is used. Importantly, immunodeficient mice bearing A431-CCK2R xenografts are previously used for the preclinical evaluation of radiolabeled minigastrin analogue pharmacokinetics, biodistribution, dosimetry or toxicity, required for regulatory approval of a phase I clinical trial in medullary thyroid cancer (MTC) patients (Maina et al., Eur J Pharm Sci. 2016; 91:236-42).

For the biodistribution study, 2×10⁶ of A431/CCKBR cells in 0.1 mL of sterile phosphate-buffered saline (PBS) are injected subcutaneously (s.c.) on the left and right flank (two tumors per animal) of anesthetized nude mice (immunocompromised CD-1 female nude mice; Charles Rivers, Germany). 12 to 14 days after implantation, nude mice carrying A431/CCKBR tumors of approximately 0.1-0.2 cm³ are intravenously injected with 38 kBq HPLC-purified ²²⁵Ac-PP-F11N (0.08 pmol) or 150 kBq HPLC-purified ¹⁷⁷Lu-PP-F11N (0.21 pmol). 1, 4, 24, 48 h and 7 days after administration of radiolabeled peptides, mice are sacrificed and the post mortem dissected tumors and organs are weighted and their activity is measured on a gamma counter (Packard Cobra II Auto Gamma, PerkinElmer, Switzerland).

For the therapy study, nude mice are subcutaneously injected with 5×10⁶ A431/CCKBR cells on the left shoulder. 5 to 7 days after tumor implantation, nude mice carrying A431/CCKBR tumors of approximately 0.1-0.2 cm³ are randomly grouped and intravenously injected with 30, 45, 60, 90 and 120 kBq of HPLC-purified ²²⁵Ac-PP-F11N in 100 μL PBS. The control group is injected with 100 μL PBS. Tumor diameters and mice weight are recorded daily during the working days. Tumor volume is calculated by using the formula formula V=(W²×L)/2 (Faustino-Rocha et al. Lab Anim. 2013, 42(6), 217-224). Nude mice are sacrificed when the tumor volume exceeded 1.5 cm³. The data are obtained from two sets of experiments including control, 30, 45, 60 kBq and control, 60, 90, 120 kBq treatment groups.

All experiments are performed in accordance with Swiss Animal Protection Laws.

### 6.2.8 SPECT/CT

For the SPECT/CT experiments, radiolabeled ¹¹¹In-PP-F11N is purified by HPLC, concentrated on SpeedVac, diluted to 13 MBq per 100 μL of PBS and intravenously injected into nude mice (13 MBq/100 μL per mouse). Imaging of ¹¹¹In-PP-F11N in A431/CCKBR tumor bearing nude mice after treatment with 30, 45 and 60 kBq of ²²⁵Ac-DOTA-PP-F11N is performed by single-photon emission computed tomography (SPECT) combined with X-ray computed tomography (multipinhole small-animal Nano SPECT/CT camera, Mediso Medical Imaging Systems). All mice are sacrificed 2 h after injections and used directly for 7.5 min CT followed by a 45 min SPECT scan. Image reconstruction and processing is accomplished by using VivoQuant 3.0 Patch1 software.

### 6.2.9 Immunochemistry

Paraffin sections of formalin-fixed A431/CCKBR tumors were subjected to deparaffinization. Rehydrated slides were pretreated in 10 mM citrate buffer, pH 6.0, at 98° C. for 60 minutes, followed by incubation with 4% fat-free milk in PBS for 90 minutes. For avidin/biotin blocker treatment (Invitrogen) and detection, the ABC method was used according to the manufacturer's instructions. For monoclonal antibody against Ki67 (Thermo Scientific, SP6) signals were recorded using an automated instrument reagent system (Discovery XT, Ventana Medical System Inc.) according to the user manual. Images of hematoxylin-counterstained sections were captured (Nikon, YTHM) and analyzed using ImageAccess Enterprise7 and ImageJ software (Schneider et al. Nat Methods 2012, 9(7), 671-675).

### 6.2.10 Bioinformatics Analysis and Statistics

GraphPad Prism 7.00 for Windows was used for all statistical analysis. Two-tailed heteroscedastic Student's t test is performed for two groups in the biodistribution study, whereas one-way ANOVA combined with Dunnett's multiple comparison test was used to compare control and all ²²⁵Ac-PP-F11N-treated groups in the therapy study. Log-rank (Mantel-Cox) test and Gehan-Breslow-Wilcoxon test are performed to compare different survival curves of treatment groups with the control group. Endpoints are defined as death in survival curves. Values of P<0.05 are considered statistically significant. The results are reported as mean±standard deviation of at least three independent replicates.

### Example 1: In Vitro Internalization and Cytotoxic Effects of ²²⁵Ac-PP-F11N

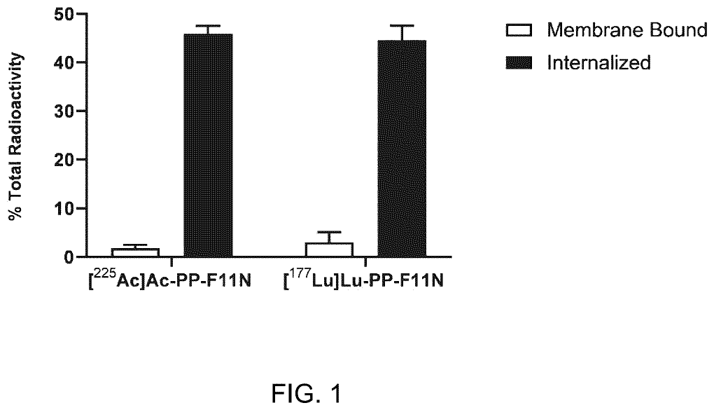

To evaluate the affinity and specificity of the alpha radiolabeled gastrin analogue of the present invention for CCKBR, the in vitro internalization assay described above was performed using purified ²²⁵Ac-PP-F11N (prepared as described above). The internalization rate of ²²⁵Ac-PP-F11N reached 45% and the membrane-bound activity was 1% of total activity. The results are depicted in FIG. 1. Presence of the blocking peptide (LEEEEEAYGWMDF)(SEQ ID NO: 1) inhibited internalization rate to 1.4%, indicating CCKBR-specific cellular uptake of ²²⁵Ac-PP-F11N. The experiment was repeated using ¹⁷⁷Lu-PP-F11N (prepared as indicated above). It was found that both compounds ²²⁵Ac-PP-F11N and ¹⁷⁷Lu-PP-F11N exhibit similar in vitro internalization rates.

To analyze the cytotoxic effect, the cell proliferation assay described above was performed. EA₅₀ value was calculated and reached 6.2±1.1 kBq/mL at 24 h after ²²⁵Ac-PP-F11N treatment (data not shown). Maximum cytotoxic effect (0% of cell viability) was reached at 100 kBq/mL.

These results demonstrate that the compound of the present invention exhibits CCKBR-specific cellular uptake (with an internalization rate of 45%) and a potent cytotoxic effect.

### Example 2: In Vivo Biodistribution of ²²⁵Ac-PP-F11N

The in vivo biodistribution of the compound of the present invention was evaluated by performing the biodistribution study described above using ²²⁵Ac-PP-F11N.

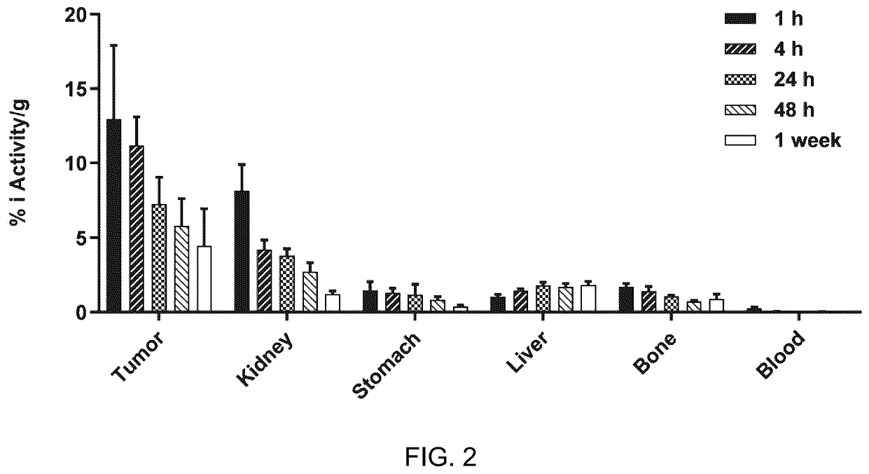

The biodistribution studies at 1, 4, 24, 48 h and 7 days post radiopharmaceutical application were performed in A431/CCKBR tumor bearing nude mice. High tumor uptake of ²²⁵Ac-PP-F11N was observed at 1 and 4 h post injection and reached 13 and 11.2% of injected activity per gram (% i.A./g), respectively (FIG. 2). After 24, 48 h and 7 days, the tumor uptake decreased as expected to 7.2, 5.7 and 4.5% i.A./g, respectively. Analysis of the healthy organs revealed 1.5% i.A./g in the stomach at 1 h post injection (p.i.) due to the endogenous expression of CCKBR. The activity in stomach remained similar within 24 hours (1.3 and 1.2% i.A./g at 4 and 24 h) and decreased to 0.8 and 0.3% i.A./g at 48 h and 7 days p.i. A 50% decrease in radiolabeled gastrin accumulation from 8.1 to 4.2% i.A./g was observed in the kidney within the first four hours. The activity level further decreased from 3.8 to 1.2% at 48 h and 1 week post injection. In other analyzed organs, the activity reached maximum 0.38 to 0.02% i.A./g at 1 h post injection and decreased within 1 week to 0.15-0.01%.

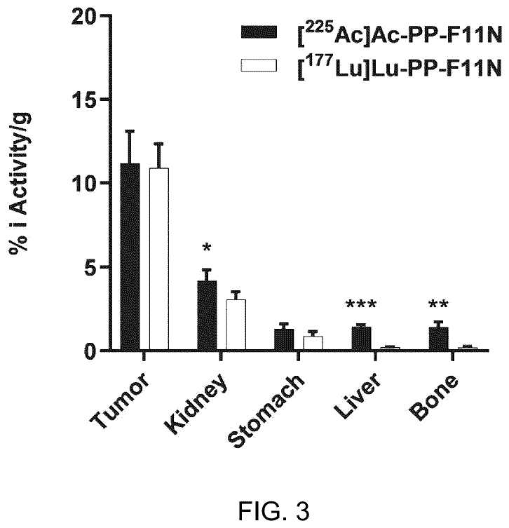

Furthermore, a comparative biodistribution study showed that ²²⁵Ac-PP-F11N had a similar biodistribution profile as lutetium-177 labeled minigastrin analogue (¹⁷⁷Lu-PP-F11N) at 4 h post injection (FIG. 3). Tumor uptake were about the same but moderate higher non-specific uptake of ²²⁵Ac-PP-F11N was observed in the liver and bone as compared to ¹⁷⁷Lu-PP-F11N and reached less than 2% i.A./g at all analyzed time points.

It was found that the compound of the present invention shows excellent biodistribution properties, i.e. an excellent tumor-to-kidney and tumor-to-stomach ratios at analyzed time points, indicating specific accumulation of ²²⁵Ac-PP-F11N in tumor tissues and low accumulation of ²²⁵Ac-PP-F11N in organs endogenously expressing CCKBR. Furthermore, no significant differences in body or organ weights, general health or anatomy were observed.

### Example 3: In Vivo Therapeutic Effects of ²²⁵Ac-PP-F11N

To evaluate the therapeutic efficacy of the compound of the present invention, the therapy study described above was performed using ²²⁵Ac-PP-F11N. Tumor growth and mean survival time of immunocompromised A431/CCKBR-tumor bearing nude mice were evaluated after administration of five different doses of purified ²²⁵Ac-PP-F11N.

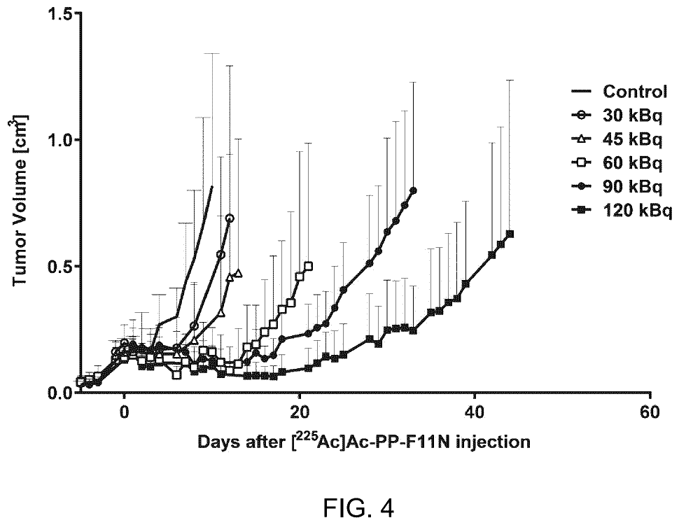

As shown in FIG. 4, treatment with ²²⁵Ac-PP-F11N significantly inhibited tumor growth in a dose dependent manner. On day 11, where all mice were still present in all groups, the average tumor volume in the control group reached 0.78 cm³, whereas average tumor size in 30, 45, 60, 90 and 120 kBq ²²⁵Ac-PP-F11N-treated mice was reduced to 0.54 (P=0.0142), 0.31 (P<0.001), 0.12 (P<0.001), 0.11 (P<0.001) and 0.07 cm³ (P<0.001), respectively. In the control group, 11 days after PBS injection, first mouse was euthanized due to exceeding the tumor volume of 1.5 cm³ (endpoint), whereas in the 30, 45, 60, 90 and 120 kBq ²²⁵Ac-PP-F11N treatment groups first tumor exceeded 1.5 cm³ on day 12, 13, 21, 33 and 44, respectively. During the treatment, no body weight losses or other cytotoxicity symptoms and the gain of the body weight was observed in all groups. Moderate increase in the body weight of the control group was observed, presumably resulting from the faster tumor growth.

Furthermore, it was observed that the treatment with ²²⁵Ac-PP-F11N increased life-span in a dose dependent manner. The mean survival time in the control group was 17 days, whereas the mean survival in 30, 45, 60, 90 and 120 kBq ²²⁵Ac-PP-F11N-treated mice was extended to 22, 27, 34, 44 and 58 days, respectively. The results are shown in Table 1 below.

TABLE 1
Mean survival times of A431/CCKBR tumor-bearing nude mice

| Treatment dose | Mean survival | |
|---|---|---|
| ²²⁵Ac-PP-F11N | (days) | P-value* |
| Control | 17 | — |
| 30 kBq | 22 | 0.36 |
| 45 kBq | 27 | 0.04 |
| 60 kBq | 34 | <0.0001 |
| 90 kBq | 44 | <0.0001 |
| 120 kBq | 58 | <0.0001 |

*P-values were acquired through comparing different survival curves of treatment groups with the control group by performing Log-rank (Mantel-Cox) test

The above results demonstrate that the compound of the present invention exhibits excellent therapeutic efficacy while adverse side effects due to accumulation of the compound in healthy tissues can be prevented.

### Example 4: Histopathological Analysis and PET/SPECT Imaging

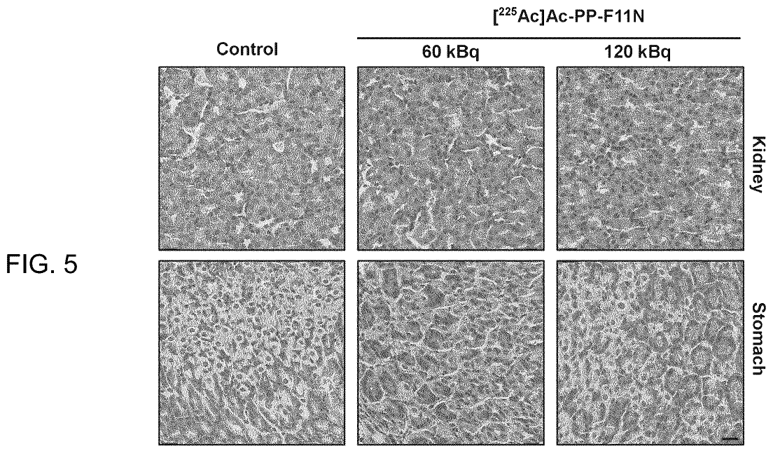

In order to further analyze the side effects (toxicity) of ²²⁵Ac-PP-F11N for the healthy organs, kidney and stomach isolated from the control and ²²⁵Ac-PP-F11N-treated mice of example 3 were stained with hematoxylin and eosin (HE) in the late stage of the therapy. The organs derived from the control group were isolated from day 11-26 after PBS injection, whereas from 60 and 120 kBq ²²⁵Ac-PP-F11N treatment groups the organs were dissected between 34 and 49 day. HE stains showed no difference among control and ²²⁵Ac-PP-F11N-treated groups (FIG. 5), indicating no signs of acute radiation toxicity during the alpha therapy in the stomach and kidneys.

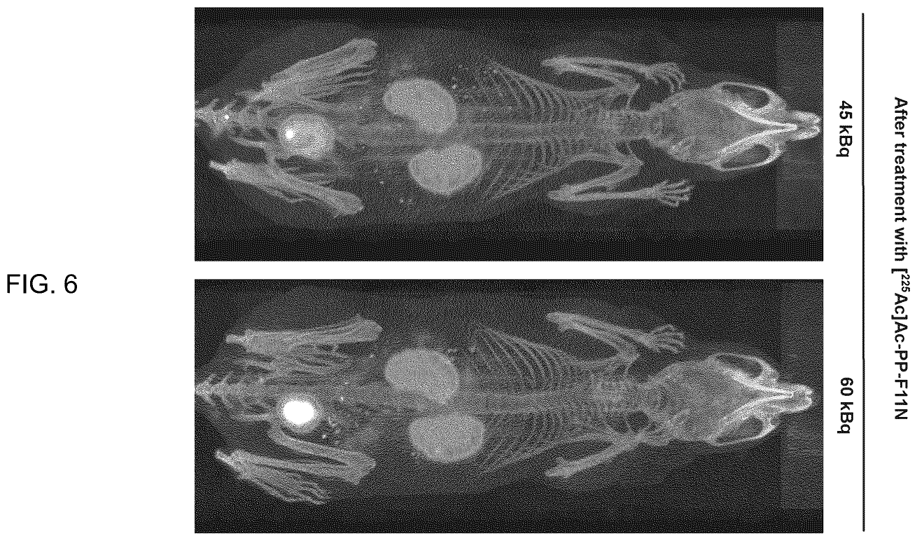

As displayed in FIG. 6, SPECT/CT images of n-PP-F11N in tumor-free mice from 45 kBq (left picture) and 60 kBq (right picture)²²⁵Ac-PP-F11N-treatment groups indicate no detectable tumors. Radioactive signals were only detected in the excretion organs including kidneys and bladder.

These results demonstrate the outstanding therapeutic efficacy achieved by the compound of the present invention as well as its low toxicity for healthy tissues, in particular for the stomach. The lack of toxicity for the stomach is surprising since the use of alpha radiolabeled gastrin compounds has been previously associated with cytotoxic side effects such as hemorrhagic gastritis (Semin Nucl Med. 2002, 32(2), 97-109). Therefore, the compound of the present invention can effectively be used in methods of treating CCKBR positive diseases, in particular CCKBR positive cancers such as MTC.

1-13. (canceled)

14. An alpha radiolabeled gastrin analogue having a formula (1) of:
(1)
(SEQ ID NO: 5)
Y-(DGlu)₆-Ala-Tyr-Gly-Trp-Axx-Asp-Phe-NH₂
wherein,
Axx represents an amino acid isosteric with methionine; and
Y represents a moiety that chelates an alpha radionuclide.

15. The alpha radiolabeled gastrin analogue according to claim 14, wherein Axx represents an amino acid selected from the group consisting of: isoleucine (Ile), norleucine (Nle), 2-amino-5-heptenoic acid, homo-norleucine (homo-Nle), homo-cysteine (homo-Cys), 2-amino-4-methoxybutanoic acid, telluromethionine (Te-Met), selenomethionine (Se-Met) and phenylglycine (Phg).

16. The alpha radiolabeled gastrin analogue according to claim 14, wherein Y is a moiety derived from 1,4,7,10-tetraazacyclododecane-1,4,7,10-tetraacetic acid (DOTA), 1,4,7,10,13,16-hexaazacyclohexadecane-1,4,7,10,13,16-hexaacetic acid (HEHA), 2-(4-isothiocyanatobenzyl)-1,4,7,10,13,16-hexaazacyclohexadecane-1,4,7,10,13,16-hexaacetic acid (HEHA-NCS), [6,6′-({9-hydroxy-1,5-bis(methoxycarbonyl)-2,4-di(pyridin-2-yl)-3,7-diazabicyclo[3.3.1]nonane-3,7-diyl}bis(methylene))dipicolinic acid] (H₂Bispa²), N,N′-bis[(6-carboxy-2-pyridil)methyl]-4,13-diaza-18-crown-6 (Macropa), 6-[[16-[(6-carboxypyridin-2-yl)methyl]-1,4,10,13-tetraoxa-7,16-diazacyclooctadec-7-yl]methyl]-4-isothiocyanatopyridine-2-carboxylic acid (Macropa-NCS), Bis(phenyliminodiacetate)-diazacrown ether (Macropid) or t-Bu-calix[4]arene tetracarboxylic acid.

17. The alpha radiolabeled gastrin analogue according to claim 14, wherein the alpha radionuclide is selected from the group consisting of: ²¹²Bi, ²¹³Bi, ²²⁵Ac, ²²⁵Fm, ²¹¹At ²²³Ra, ¹⁴⁹Tb, ²¹²Pb, ²²⁶Th and ²²⁷Th.

18. The alpha radiolabeled gastrin analogue according to claim 14, wherein the alpha radionuclide is ²²⁵Ac.

19. The alpha radiolabeled gastrin analogue according to claim 14, wherein the alpha radionuclide satisfies at least one of the following (i) and (ii):
(i) a linear energy transfer (LET) of 20 keV/μm to 190 keV/μm; and
(ii) a tissue penetration range (TPR) of 20 μm to 150 μm.

20. The alpha radiolabeled gastrin analogue according to claim 14, wherein the alpha radiolabeled gastrin analogue is represented by a formula (2):
(2)
(SEQ ID NO: 6)
Y-(DGlu)₆-Ala-Tyr-Gly-Trp-Nle-Asp-Phe-NH₂
wherein Y represents the moiety derived from 1,4,7,10-tetraazacyclododecane-1,4,7,10-tetraacetic acid (DOTA) that chelates ²²⁵Ac.

21. The alpha radiolabeled gastrin analogue according to claim 14, wherein Y is the moiety derived from 1,4,7,10-tetraazacyclododecane-1,4,7,10-tetraacetic acid (DOTA), or 1,4,7,10,13,16-hexaazacyclohexadecane-1,4,7,10,13,16-hexaacetic acid (HEHA).

22. The alpha radiolabeled gastrin analogue according to claim 14, wherein the alpha radionuclide is selected from the group consisting of: ²¹³Bi, ²²⁵Ac, and ¹⁴⁹Tb.

23. The alpha radiolabeled gastrin analogue according to claim 14, wherein the alpha radionuclide satisfies at least one of the following (i) and (ii):
(i) a linear energy transfer (LET) of 80 keV/μm; and
(ii) a tissue penetration range (TPR) of 40 μm to 100 μm.

24. A method of treating at least one cholecystokinin B (CCKB) receptor positive disease, which comprises the step of:
administering a therapeutically effective dose of an alpha radiolabeled gastrin analogue to a human subject diagnosed with the at least one CCKB receptor positive disease, wherein the alpha radiolabeled gastrin analogue is as defined b the formula:
(1)
(SEQ ID NO: 5)
Y-(DGlu)₆-Ala-Tyr-Gly-Trp-Axx-Asp-Phe-NH₂
wherein,
Axx represents an amino acid isosteric with methionine; and
Y represents a moiety that chelates an alpha radionuclide.

25. The method according to claim 24, wherein the alpha radiolabeled gastrin analogue is represented by the following formula (2):
(2)
(SEQ ID NO: 6)
Y-(DGlu)₆-Ala-Tyr-Gly-Trp-Nle-Asp-Phe-NH₂
wherein Y represents a moiety derived from 1,4,7,10-tetraazacyclododecane-1,4,7,10-tetraacetic acid (DOTA) that chelates ²²⁵Ac.

26. The method according to claim 24, wherein the at least one CCKB receptor positive disease is selected from the group consisting of: gastric cancer (GC), pancreatic adenocarcinoma (PADC), small-cell lung cancer (SCLC), extrapulmonary small-cell carcinoma (EPSCC), medullary thyroid cancer (MTC), gliomas, gastroenteropancreatic neuroendocrine tumors (GEP-NETs), colon cancer, ovarian cancer, breast cancer, and any CCKB receptor positive cancer or tumors.

27. The method according to claim 24, wherein the at least one CCKB receptor positive disease is selected from small-cell lung cancer (SCLC), extrapulmonary small-cell carcinoma (EPSCC) and medullary thyroid cancer (MTC).

28. The method according to claim 24, wherein the therapeutically effective dose that is administered to the human subject is of from 10 to 40,000 kBq/kg.

29. The method according to claim 24, wherein the at least one CCKB receptor positive disease is medullary thyroid cancer (MTC).

30. The method according to claim 24, wherein the therapeutically effective dose that is administered to the human subject is of from 30 to 1,000 kBq/kg.

31. The method according to claim 24, wherein the therapeutically effective dose that is administered to the human subject is of from 50 to 200 kBq/kg.

32. A method for treating cholecystokinin B (CCKB) receptor positive diseases, which comprises the step of:
administering a therapeutically effective dose of an alpha radiolabeled gastrin analogue to a human subject in need thereof, wherein the alpha radiolabeled gastrin analogue is as defined by the formula:
(2)
(SEQ ID NO: 6)
Y-(DGlu)₆-Ala-Tyr-Gly-Trp-Nle-Asp-Phe-NH₂
wherein Y represents the moiety derived from 1,4,7,10-tetraazacyclododecane-1,4,7,10-tetraacetic acid (DOTA) that chelates ²²⁵Ac.


SEQUENCE LISTING (7 sequences)

SEQ ID NO: 1 - protein, 13 aa, Homo sapiens
  misc_feature: minigastrin (blocking peptide)
  MOD_RES (13)..(13): AMIDATION
LEEEEEAYGWMDF

SEQ ID NO: 2 - protein, 5 aa, Artificial Sequence
  C-terminal pentapeptide analoguous to the C-terminal amino acid sequence found in CCKBR-binding endogenous peptide hormones
  VARIANT (3)..(3): Represents an amino acid isosteric with methionine
  MOD_RES (5)..(5): AMIDATION
GWXDF

SEQ ID NO: 3 - protein, 17 aa, Homo sapiens
  misc_feature: minigastrin as described on page 8 of the application as filed
  MOD_RES (17)..(17): AMIDATION
LEEEEEEEEEAYGWMDF

SEQ ID NO: 4 - protein, 13 aa, Artificial Sequence
  Synthetic
  MOD_RES (1)..(1): 1,4,7,10-tetraazacyclododecane-1,4,7,10- tetraacetic acid (DOTA)
  SITE (1)..(6): D-Glu
  VARIANT (11)..(11): Nle
  MOD_RES (13)..(13): AMIDATION
EEEEEEAYGWXDF

SEQ ID NO: 5 - protein, 13 aa, Artificial Sequence
  Synthetic
  SITE (1)..(1): contains a moiety that chelates an alpha radionuclide
  SITE (1)..(6): D-Glu
  VARIANT (11)..(11): an amino acid isosteric with methionine
  MOD_RES (13)..(13): AMIDATION
EEEEEEAYGWXDF

SEQ ID NO: 6 - protein, 13 aa, Artificial Sequence
  Synthetic
  SITE (1)..(1): contains a moiety derived from DOTA that chelates 225Ac
  SITE (1)..(6): D-Glu
  VARIANT (11)..(11): Nle
  MOD_RES (13)..(13): AMIDATION
EEEEEEAYGWXDF

SEQ ID NO: 7 - protein, 13 aa, Artificial Sequence
  Synthetic
  SITE (1)..(6): D-Glu
  VARIANT (11)..(11): an amino acid isosteric with methionine
  MOD_RES (13)..(13): AMIDATION
EEEEEEAYGWXDF